(19) United States
(12) Patent Application Publication
(10) Pub. No.: US 2023/0210621 A1
(43) Pub. Date: Jul. 6, 2023

(54) SAFETY HAND SENSOR SYSTEMS FOR ROBOTIC SURGICAL SYSTEM

(71) Applicant: EndoQuest Robotics, Inc., Houston, TX (US)

(72) Inventors: Jinseok Noh, Houston, TX (US); Seungwoo Park, Houston, TX (US); Jihwan Jung, Houston, TX (US); Jiwon Choi, Houston, TX (US); Seoungkyou Lee, Houston, TX (US); Dongsuk Shin, Houston, TX (US)

(73) Assignee: EndoQuest Robotics, Inc., Houston, TX (US)

(21) Appl. No.: 18/122,018

(22) Filed: Mar. 15, 2023

Related U.S. Application Data
(63) Continuation of application No. PCT/US2022/051237, filed on Nov. 29, 2022.
(60) Provisional application No. 63/284,191, filed on Nov. 30, 2021.

(51) Int. Cl. A61B 34/00; A61B 34/37; B25J 13/02

(52) U.S. Cl. CPC A61B 34/74; A61B 34/37; B25J 13/02; A61B 2034/742

(57) ABSTRACT

In accordance with at least one aspect of this disclosure, a user input handle for a hand control device of a robotic surgical system can include a gripping portion configured to be grasped by a user. The gripping portion can include a grip safety sensor configured to sense whether the gripping portion is being grasped by a user.

### CROSS-REFERENCE TO RELATED APPLICATIONS

This application is a continuation of International Patent Application No. PCT/US2022/051237 filed Nov. 29, 2022, which claims priority to and the benefit of U.S. Provisional Application No. 63/284,191, filed Nov. 30, 2021, the entire contents of which are herein incorporated by reference in their entirety.

### FIELD

This disclosure relates to robotic surgical systems, e.g., for minimally invasive surgery including, but not limited to, endoluminal and single-site surgery.

### BACKGROUND

Minimally invasive surgery such as endoluminal and single-site robotic surgery offer significant advantages versus traditional robotic surgery. For example, in endoluminal robotic surgery, no incision need be made to access difficult to access locations within a patient's natural lumen. This dramatically reduces and/or eliminates recovery time and improves procedural safety. A single-site system reduces incisions to a minimum single-site, which reduces an otherwise larger number of incisions to provide access for certain procedures.

Certain endoluminal and single-site robotic surgical systems have been proposed. Examples of such systems and related components can be found in U.S. Pat. No. 10,881,422, as well as U.S. Patent Application Nos. US20210322046, US20210322045, US20190117247, US20210275266, US20210267702, US20200107898, US20200397457, US202000397456, US20200315645, and US201962914226, all of the above being incorporated by reference herein in their entirety.

Conventional surgical robotics and systems have generally been considered satisfactory for their intended purpose. However, there is still a need in the art for improved robotic surgical systems, devices, methods, controls, and components, especially those configured for endoluminal and single-site surgery. The present disclosure provides improvements in such areas, for example.

### SUMMARY

In accordance with at least one aspect of this disclosure, a user input handle for a hand control device of a robotic surgical system can include a gripping portion configured to be grasped by a user. The gripping portion can include a grip safety sensor configured to sense whether the gripping portion is being grasped by a user and to output a safety signal to indicate whether the gripping portion is being grasped by a user.

The grip safety sensor can be positioned on the gripping portion to be in contact with at least a portion of the user's hand when the user is grasping the handle. For example, the grip safety sensor can be configured to output the safety signal when detected to indicate to a control module that the gripping portion is properly grasped by the user. Any suitable sensor type is contemplated herein (e.g., a proximity sensor, powered, or unpowered, etc).

In certain embodiments, the handle can include one or more finger levers configured to receive a finger of the user to provide an additional output from the handle (e.g., to mimic the shape and function of an endoscopic tool handle). In certain embodiments, the gripping portion can include a pistol grip shape. In certain embodiments, the handle can also include one or more finger clutch buttons configured to be actuated by a finger of the user when grasping the handle to provide a clutch output from the handle. In certain embodiments, the gripping portion can include the one or more finger clutch buttons. In certain embodiments, the grip safety sensor can be a button that is positioned on the gripping portion to be in contact with a palm of the user's hand when the user is grasping the handle.

In certain embodiments, the gripping portion can be configured to be detachable from the hand control device of the robotic surgical system. Any other suitable configuration is contemplated herein.

In accordance with at least one aspect of this disclosure, a hand control system for a robotic surgical system can include one or more hand control devices configured to be operated to control one or more surgical instruments. The system can include a user input handle connected to each hand control device. The user input handle can be any suitable handle as disclosed herein (e.g., as described above). The system can include a control module configured to receive the safety signal to activate a control mode, receive input signals from the one or more hand control devices to control the one or more the one or more surgical instruments in the control mode, and activate a safety mode to disable control of the surgical instruments using the one or more hand control devices when the safety signal is not received. Any other suitable safety signal control logic (e.g., due to a lack of a signal, a second signal, etc.) to switch between the control mode and the safety mode is contemplated herein.

In accordance with at least one aspect of this disclosure, a robotic surgical system can include one or more hand control devices configured to be operated to control one or more surgical instruments, and a user input handle connected to each hand control device. The user input handle can include a gripping portion configured to be grasped by a user. The gripping portion can include a grip safety sensor configured to sense whether the gripping portion is being grasped by a user and to output a safety signal to indicate whether the gripping portion is being grasped by a use. The robotic surgical system can include any suitable portions of a hand control system as disclosed herein, e.g., as described above.

These and other features of the embodiments of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 is a perspective view of an embodiment of a user input handle in accordance with this disclosure;

FIG. 2 is a perspective view of the embodiment of FIG. 1 associated with a user console of a robotic surgical system;

FIG. 3 is a perspective view showing a user grasping the embodiment of FIG. 1;

FIG. 4 illustrates a user operating two hand control devices using the embodiment of a handle of FIG. 1 on each device;

FIG. 5 is a schematic view of an embodiment of a control system in accordance with this disclosure;

FIG. 6A is a perspective view of an embodiment of a handle in accordance with this disclosure;

FIG. 6B is a right side elevation view of the embodiment of FIG. 6A;

FIG. 6C is a front elevation view of the embodiment of FIG. 6A;

FIG. 6D is a left side elevation view of the embodiment of FIG. 6A;

FIG. 6E is a rear elevation view of the embodiment of FIG. 6A;

FIG. 6F is a bottom plan view of the embodiment of FIG. 6A;

FIG. 6G is a top plan view of the embodiment of FIG. 6A;

FIG. 6H is a perspective view of the embodiment of FIG. 6A, shown having a housing cover portion removed;

FIG. 6I is a perspective view of the embodiment of FIG. 6A, shown having a main housing portion in isolation; and

FIG. 7 is a perspective view of an embodiment of a hand control device, shown having the embodiment of a handle of FIG. 6A attached thereto.

### DETAILED DESCRIPTION

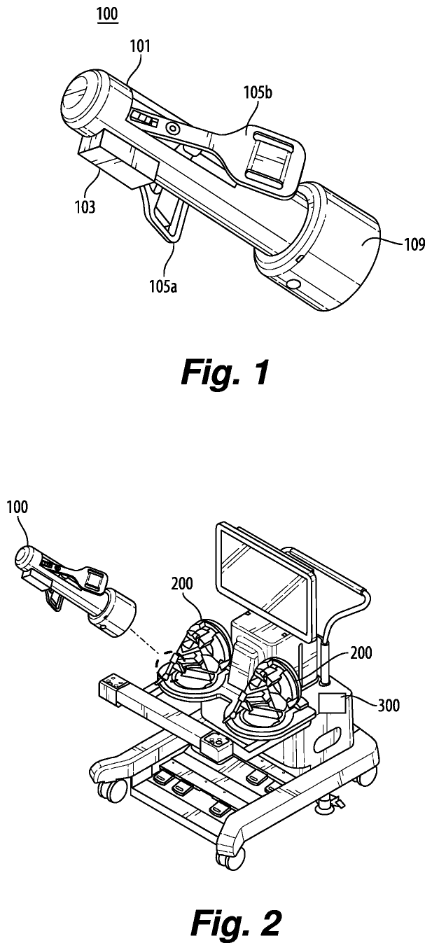

Reference will now be made to the drawings wherein like reference numerals identify similar structural features or aspects of the subject disclosure. For purposes of explanation and illustration, and not limitation, an illustrative view of an embodiment of a system in accordance with the disclosure is shown in FIG. 1 and is designated generally by reference character 100. Other embodiments and/or aspects of this disclosure are shown in FIGS. 2-7.

In accordance with at least one aspect of this disclosure, referring to FIGS. 1-4, a user input handle 100 for a hand control device 200 of a robotic surgical system (e.g., an endoluminal surgical device) can include a gripping portion 101 configured to be grasped by a user (e.g., a surgeon). The gripping portion 101 can include a grip safety sensor 103 configured to sense whether the gripping portion 101 is being grasped by a user. The sensor 103 can be configured to output a safety signal to indicate whether the gripping portion 101 is being grasped by a user.

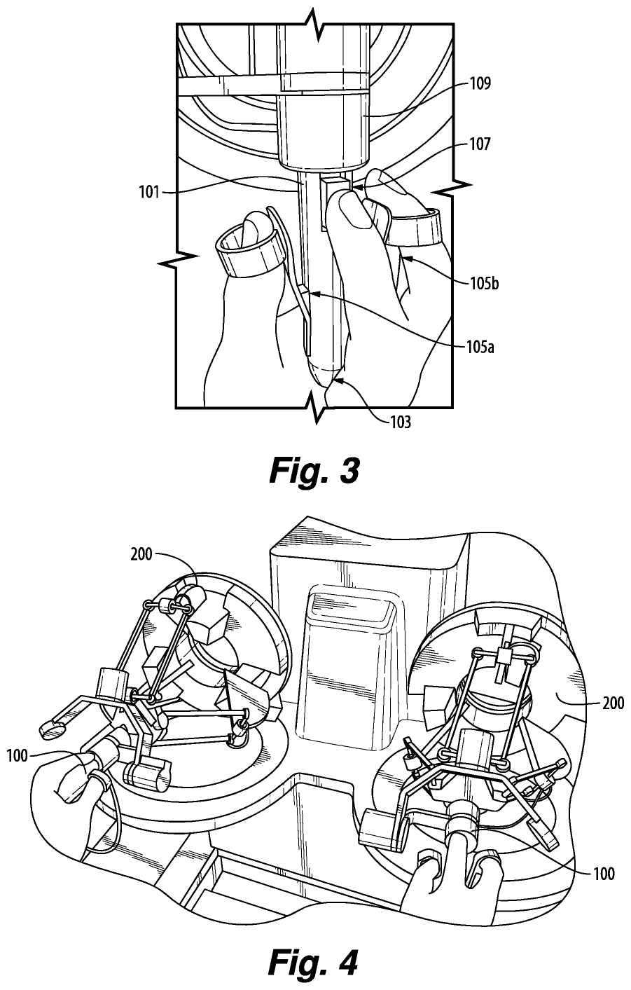

The grip safety sensor 103 can be a button (e.g., as shown in FIG. 1) that is positioned on the gripping portion 100 to be in contact with at least of a portion (e.g. a palm) of the user's hand when the user is grasping the handle 100 (e.g., as shown in FIGS. 3 and 4). For example, the grip safety sensor 103 can be configured to output the safety signal when detected to indicate to a control module 300 that the gripping portion 101 is properly grasped by the user. Any suitable sensor type is contemplated herein (e.g., a proximity sensor, powered, or unpowered, etc).

In certain embodiments, e.g., as shown in FIGS. 1-4, the handle 100 can include one or more finger levers 105a, 105b configured to receive a finger of the user to provide an additional output (e.g., a gripping function for jaws of an endoscopic tool) from the handle 100 (e.g., to mimic the shape and function of an endoscopic tool handle). In certain embodiments, the handle 100 can also include one or more finger clutch buttons 107 configured to be actuated by a finger of the user when grasping the handle 100 to provide a clutch output (e.g., for selectively disengaging instrument control) from the handle 100.

In certain embodiments, the gripping portion 101 can be configured to be detachable from the hand control device 200 of the robotic surgical system. For example, the gripping portion 101 can include a fitting 109 (e.g., a threaded connection, a clip connection, a screw on connection, etc.) configured to attach to the hand control device 200. Any other suitable configuration is contemplated herein.

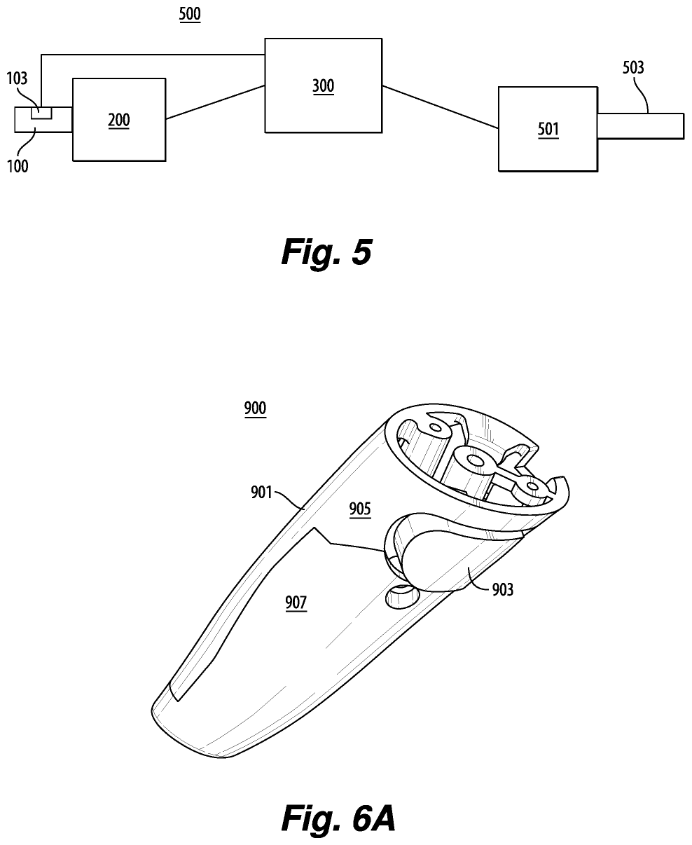

In accordance with at least one aspect of this disclosure, referring to FIG. 5, a control system 500 for a robotic surgical system can include one or more hand control devices 200 configured to be operated to control one or more surgical instruments 503 (e.g., via robotic instrument controller 501). The system 500 can include a user input handle 100 connected to each hand control device 200. The user input handle 100 can be any suitable handle as disclosed herein (e.g., handle 100 as described above). The system 500 can include a control module 300 configured to receive the safety signal to activate a control mode, receive input signals from the one or more hand control devices 200 to control the one or more the one or more surgical instruments 503 in the control mode, and activate a safety mode to disable control of the surgical instruments 503 using the one or more hand control devices 200 when the safety signal is not received. For example, the safety signal can be a change in signal state (e.g., from on to off, or off to on), and/or can be intermittent, continuous, or singular. Any suitable signal is contemplated herein. Any other suitable safety signal control logic (e.g., due to a lack of a signal or state change of a signal, a second signal, etc.) to switch between the control mode and the safety mode is contemplated herein. In accordance with at least one aspect of this disclosure, a robotic surgical system can include one or more embodiments of a hand control device and a user input as disclosed herein, e.g., as described above.

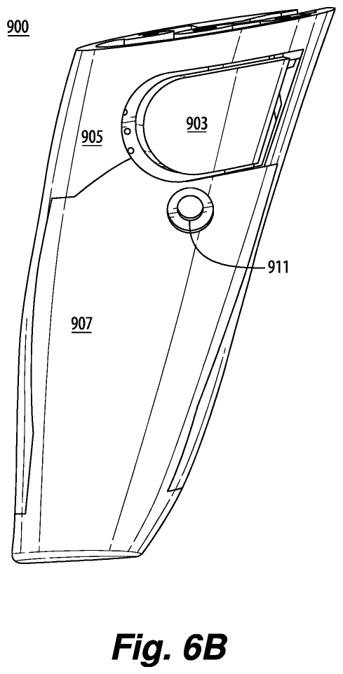

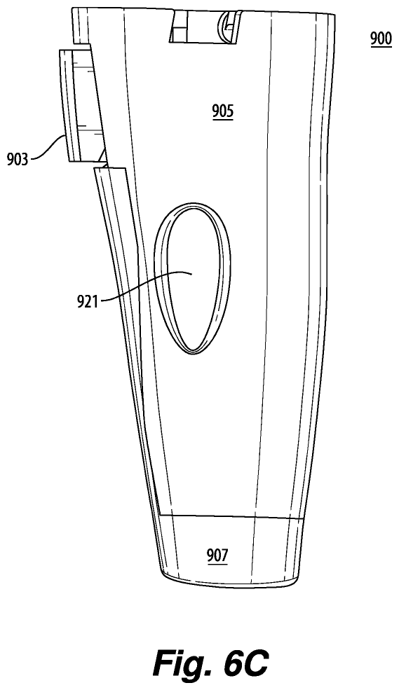

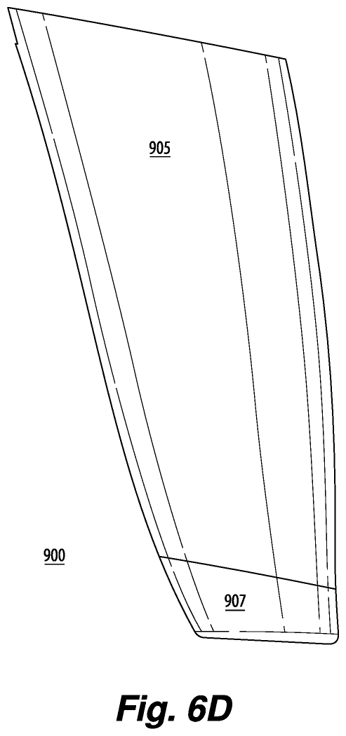

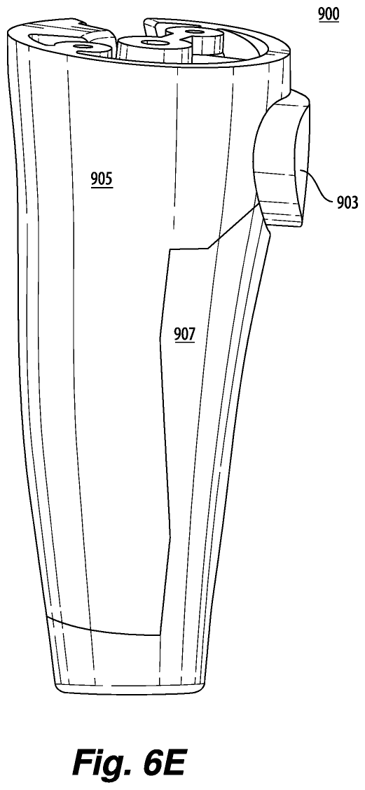

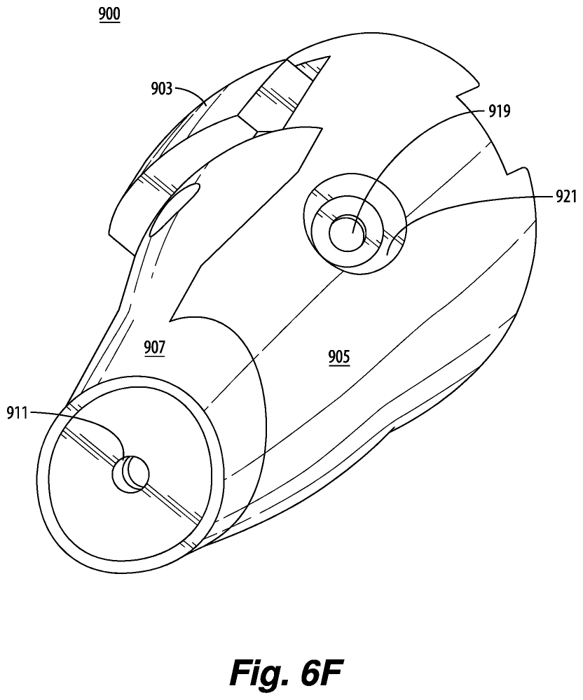

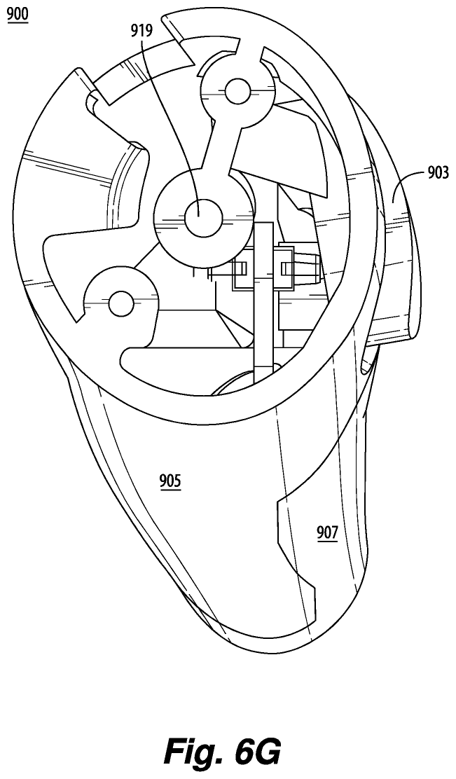

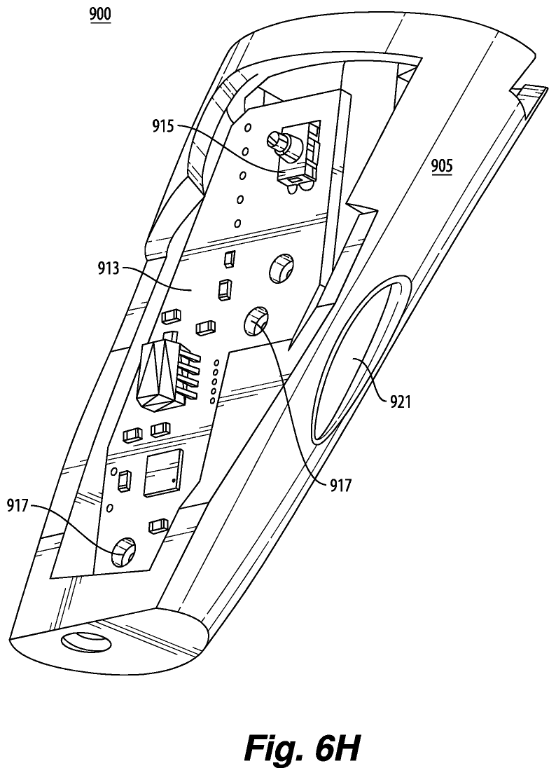

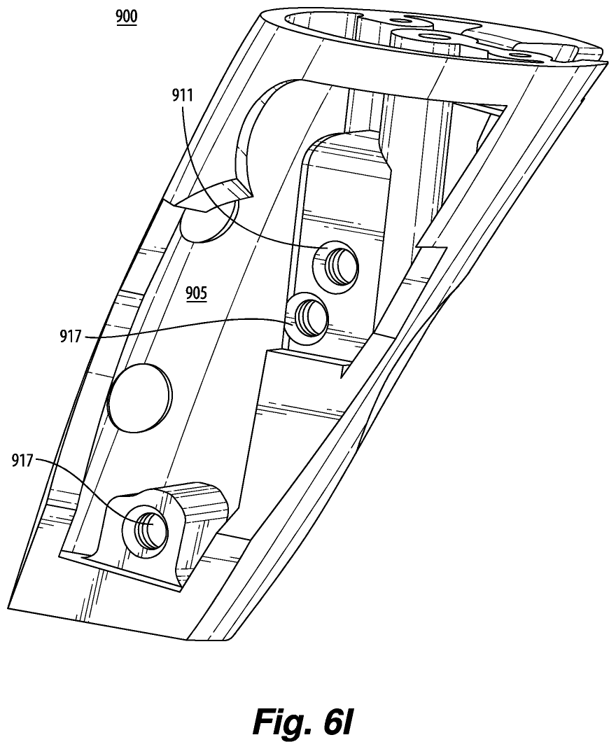

Referring to FIGS. 6A-6I, in certain embodiments, a handle 900 can include a gripping portion 901 that has a pistol grip shape. In certain embodiments, the handle 900 can also include one or more finger clutch buttons 903 configured to be actuated by a finger of the user when grasping the handle 900 to provide a clutch output from the handle 900. In certain embodiments, the gripping portion can include the one or more finger clutch buttons 903.

For example, as shown, a finger clutch button 903 can be located near a top of the handle 900 and in such a position that a thumb of a user can actuate the finger clutch button 903 when the handle 900 is being gripped by a user (e.g., a left hand gripping the handle 900 as shown). This can allow selective actuation of the finger clutch button 903 without the user having to lift their grip or change hand position.

The gripping portion 901 can be formed by a main housing portion 905 and a housing cover portion 907 configured to connect to the main housing portion 905, e.g., via one or more fasteners (e.g., screws), e.g., via one or more fastener holes 911. The finger clutch button 903 can be mounted to be movable relative to the gripping portion 901. The finger clutch button 903 can be positioned to be laterally actuated, e.g., at or near a top of the handle 900.

The handle 900 can include a circuit board 913 (e.g., as shown in FIG. 6H) configured to fit between the main housing portion 905 and the housing cover portion 907. For example, the circuit board 913 can include a conforming shape (e.g., a step shape) configured to fit within a cavity within the handle 900. The circuit board 913 can include any suitable logic module(s) having any suitable hardware module(s) and/or software module(s) configured to enable the hand safety sensor and/or clutch button functionality, e.g., as described below. For example, the circuit board 913 can include a button switch 915 configured to be depressed by the finger clutch button 903. The circuit board 913 can include logic to sense a position of the button switch 915 and to output a clutch signal to a control system and/or to not output a control signal to the control system. Any other suitable function of the circuit board 913 is contemplated herein. The circuit board 913 can be mounted to the main housing portion 905 via one or more fasteners (e.g., screws), e.g., via one or more fastener holes 917.

In certain embodiments, the finger clutch button 903 can be the grip safety sensor (e.g., similar to sensor 103 disclosed above). In certain embodiments, the finger clutch buttons 903 can be configured to only allow control of a respective medical instrument when actively depressed. In this regard, the finger clutch button 903 can be configured to require the user to hold the finger clutch button 903 in a depressed position to operate a respective medical instrument. The finger clutch button 903 can be biased in a direction opposite of the depressed position, e.g., such that the finger clutch button 903 is biased to cause the system to disengage a respective medical instrument when the finger clutch button 903 is not depressed. Accordingly, the finger clutch button 903 can act as a manual/mechanical grip safety sensor.

In certain embodiments, the finger clutch button 903 can operate similarly to finger clutch button 107 and can require a user to press the finger clutch button 903 to disengage hand control. In such embodiments, the gripping portion 901 can include another grip safety sensor (not shown in FIGS. 6A-6I), e.g., similar to grip safety sensor 103 as disclosed herein, e.g., as described above (e.g., a palm sensor/button on the rear of the pistol grip).

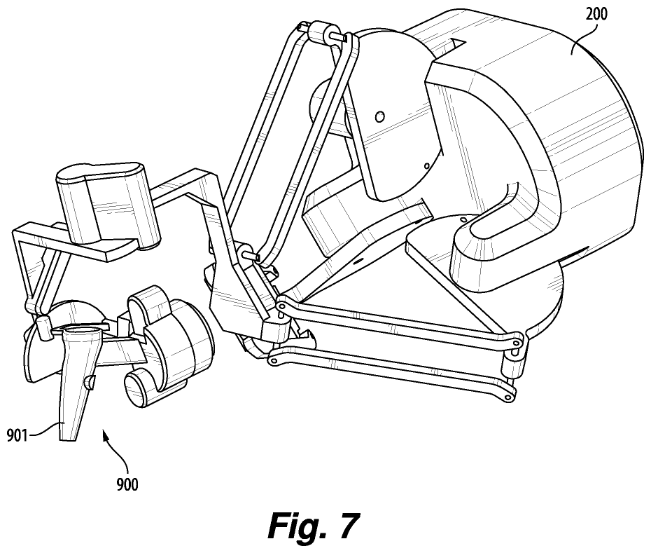

Referring additionally to FIG. 7, the handle 900 can be utilized on a hand control device 200, e.g., as disclosed above. The handle 900 can be removably attached to the hand control device 200 via one or more fasteners (e.g., a screw). For example, the handle can include a control device fastener hole 919 (e.g., defined through the main housing portion 905) configured to extend to a top of the handle 900. The control device fastener hole 919 can be deposed within a recess 921 to be hidden and/or not on an exterior surface of the handle 900 to avoid hand contact. For example, the recess 921 can be elongated in the vertical direction to require use of an elongated tool (e.g., a screwdriver) to reach the fastener. The user can align the handle 900 (e.g., via one or more alignment holes, e.g., as shown in FIG. 6G) and insert a fastener into the hole 919. The user can use a tool to affix the fastener to the control device 200. Other embodiments of handle arrangements are contemplated herein.

As disclosed above, a user input handle can include a gripping portion configured to be grasped by a user. The gripping portion can include a grip safety sensor configured to sense whether the gripping portion is being grasped by a user and to output a safety signal to indicate whether the gripping portion is being grasped by a use. A robotic surgical system can include any suitable portions of a hand control system as disclosed herein, e.g., as described above.

Embodiments can include a finger clutch button, for example. In certain embodiments, a user can press and hold the finger clutch button to hold the position and pose of the surgical instrument connected to the respective hand control device (regardless of the motion of that hand control device). For example, in certain embodiments, the motion of the hand control device will not be relayed to the respective surgical instruments when the finger clutch button is pressed and held. The user can use the finger clutch button to reposition the hand control device without changing the instrument pose for easy maneuvering of the workspace, for example.

Embodiments can include one or more grip safety sensors on or within a hand control device which can detect user errors and prevent unintentional movement, for example. Embodiments can have a safety hand sensor on a gripping portion of a control device on a surgeon console that detects unintended hand movements and can cause shutdown of instrument controller/positioning system movements (or any other suitable movement of any suitable device linked to the hand control devices 200). In certain embodiments, the finger clutch button can act as the grip safety sensor, for example, the finger clutch button can be configured to require the user to press and hold the button to enable movement of a surgical instrument, and letting go of the finger clutch button and stop user input from the hand control device from controlling the surgical instrument.

As will be appreciated by those skilled in the art, aspects of the present disclosure may be embodied as a system, method, or computer program product. Accordingly, aspects of this disclosure may take the form of an entirely hardware embodiment, an entirely software embodiment (including firmware, resident software, micro-code, etc.), or an embodiment combining software and hardware aspects, all possibilities of which can be referred to herein as a “circuit,” “module,” or “system.” A “circuit,” “module,” or “system” can include one or more portions of one or more separate physical hardware and/or software components that can together perform the disclosed function of the “circuit,” “module,” or “system”, or a “circuit,” “module,” or “system” can be a single self-contained unit (e.g., of hardware and/or software). Furthermore, aspects of this disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of this disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of this disclosure may be described above with reference to flowchart illustrations and/or block diagrams of methods, apparatus (systems) and computer program products according to embodiments of this disclosure. It will be understood that each block of any flowchart illustrations and/or block diagrams, and combinations of blocks in any flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in any flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the subject disclosure includes reference to certain embodiments, those skilled in the art will readily appreciate that changes and/or modifications may be made thereto without departing from the spirit and scope of the subject disclosure.

What is claimed is:

1. A user input handle for a hand control device of a robotic surgical system, comprising:
a gripping portion configured to be grasped by a user, wherein the gripping portion includes a grip safety sensor configured to sense whether the gripping portion is being grasped by a user and to output a safety signal to indicate whether the gripping portion is being grasped by a user.

2. The handle of claim 1, wherein the grip safety sensor is positioned on the gripping portion to be in contact with at least of a portion of the user's hand when the user is grasping the handle.

3. The handle of claim 2, wherein the grip safety sensor is configured to output the safety signal when detected to indicate to a control module that the gripping portion is properly grasped by the user.

4. The handle of claim 3, further comprising one or more finger levers configured to receive a finger of the user to provide an additional output from the handle.

5. The handle of claim 1, wherein the gripping portion includes a pistol grip shape.

6. The handle of claim 5, wherein the gripping portion includes one or more finger clutch buttons configured to be actuated by a finger of the user when grasping the handle to provide a clutch output from the handle.

7. The handle of claim 1, wherein the grip safety sensor is a button that is positioned on the gripping portion to be in contact with a palm of the user's hand when the user is grasping the handle.

8. A hand control system for a robotic surgical system, comprising:
one or more hand control devices configured to be operated to control one or more surgical instruments; and
a user input handle connected to each hand control device, the user input handle including a gripping portion configured to be grasped by a user, wherein the gripping portion includes a grip safety sensor configured to sense whether the gripping portion is being grasped by a user and to output a safety signal to indicate whether the gripping portion is being grasped by a user.

9. The system of claim 8, wherein the grip safety sensor is positioned on the gripping portion to be in contact with at least of a portion of the user's hand when the user is grasping the handle.

10. The system of claim 9, wherein the grip safety sensor is configured to output the safety signal when detected to indicate to a control module that the gripping portion is properly grasped by the user.

11. The system of claim 10, further comprising one or more finger levers configured to receive a finger of the user to provide an additional output from the handle.

12. The system of claim 8, wherein the gripping portion includes a pistol grip shape.

13. The system of claim 12, wherein the gripping portion includes one or more finger clutch buttons configured to be actuated by a finger of the user when grasping the handle to provide a clutch output from the handle.

14. The system of claim 8, further comprising a control module configured to:
receive the safety signal to activate a control mode;
receive input signals from the one or more hand control devices to control the one or more the one or more surgical instruments in the control mode; and
activate a safety mode to disable control of the surgical instruments using the one or more hand control devices when the safety signal is not received.

15. The system of claim 8, wherein the grip safety sensor is a button that is positioned on the gripping portion to be in contact with a palm of the user's hand when the user is grasping the handle.
